(19) United States
(12) Patent Application Publication
(10) Pub. No.: US 2011/0294684 A1
(43) Pub. Date: Dec. 1, 2011

(54) GENE EXPRESSION SIGNATURES FOR LUNG CANCERS

(75) Inventors: Florent Baty, Wittenbach (CH); Martin Buess, Basel (CH); Martin Brutsche, Niederteufen (CH); Martin Schumacher, Basel (CH); Sergio Kaiser, Riehen (CH); Wolfgang Budach, Schliengen (DE)

(73) Assignee: KANTON BASEL-STADT REPRESENTED BY THE UNIVERSITY HOSPITAL BASEL

(21) Appl. No.: 13/000,329

(22) PCT Filed: Jun. 19, 2009
(86) PCT No.: PCT/IB2009/006212

§ 371 Date: Aug. 22, 2011

(30) Foreign Application Priority Data
Jun. 20, 2008 (GB) 0811413.4

(51) Int. Cl. C40B 30/04; C40B 40/08; C40B 40/06; C12Q 1/68

(52) U.S. Cl. 506/9; 435/6.12; 435/6.11; 506/17; 506/16

(57) ABSTRACT

The inventors have found a group of genes whose expression in small bronchoscopic tumor samples gives significant predictions of survival. 10 of the 13 genes are indicators of risk, while the other 3 are indicators of survival.

This application claims the benefit of United Kingdom patent application 0811413.4, filed 20 Jun. 2008, the complete contents of which are incorporated herein by reference.

### TECHNICAL FIELD

This invention relates to diagnostics (and in particular prognostics) for lung cancers, such as non-small-cell lung cancers, based on the detection of biomarkers.

### BACKGROUND ART

High-throughput gene expression technology has been used to identify gene classifiers of lung cancer subtypes [1,2] or predictors for disease outcome [3]. These studies yielded an important contribution regarding the identification of distinct sub-groups among adenocarcinomas [1,3] and squamous-cell carcinomas [4,5]. These sub-categories were associated with specific gene expression patterns that correlated with survival [2]. Recent studies described gene signatures predicting survival with a good accuracy after validation in independent data sets [6] but, in contrast to breast cancer [7], clinical studies investigating the utility of prognostic gene signatures for the stratification of patients with non-small cell lung cancer (NSCLC) have started only recently.

Almost all gene expression microarray studies published so far are based on tumor samples obtained during lung cancer surgery with curative intent, and so they focus on early stages of NSCLC. As the fraction of patients undergoing surgery for lung cancer can be as low as 7% of patients with NSCLC [8], though, the findings from these studies might not reflect the whole spectrum of NSCLC patients, and is particularly scarce for patients with advanced NSCLC.

Spira et al. [9] recently evaluated the diagnostic value of functional genomics of bronchial airway epithelial cells obtained with an endoscopic cytobrush in smokers with suspicion of lung cancer. They identified gene expression biomarkers based on 80 genes and these biomarkers could identify patients with lung cancer with a sensitivity and specificity of 80 and 84%, respectively.

It is an object of the invention to provide further and improved biomarkers for gene expression profiling of lung tissue for the refinement of tumor diagnosis, and in particular the prediction of survival periods. It is a further object to provide methods of prognosis that can easily be accommodated alongside techniques that are already used in current diagnostic procedures.

### DISCLOSURE OF THE INVENTION

The inventors have found 13 genes whose expression in small bronchoscopic tumor samples gives significant predictions of the duration of patient survival with an overall prognostic accuracy of 83%. The signature has been validated in four independent data sets. 10 of the 13 genes are indicators of risk, while the other 3 are indicators of survival. The signature was particularly good for identifying patients with a survival of less than one year.

An individual gene within the group of 13 can be analyzed in isolation, and this single analysis has the potential to provide useful prognostic information, but it is preferred that a combination of 2 or more of the genes (e.g. 2, 3, 4, 5, 6, 7, 8, 9, 10, 11, 12 or all 13) is analyzed.

Thus the invention provides a method of prognosis of a lung cancer in a patient, comprising a step of measuring the expression level/s, in a lung tissue sample from the patient, of one or more of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.

The method will typically include a further step of comparing the measured expression level/s to a control level in order to find if expression is up-regulated, down-regulated or unchanged, and thereby to predict if patient survival is increased or decreased relative to the control. The choice of control sample determines the information that the comparison reveals. For example, if the control level is the average expression level seen in samples taken from a population of lung cancer patients then the comparison can indicate survival duration relative to the average survival duration of that population. An aggregate increase in expression level/s for gene/s (i) to (x) in the sample indicate/s a decreased survival duration relative to the control. An aggregate decrease in expression level/s, or no change, for gene/s (i) to (x) in the sample indicate/s an increased survival duration relative to the control. An aggregate increase in expression level/s, or no change, for gene/s (xi) to (xiii) in the sample indicate/s an increased survival duration relative to the control. An aggregate decrease in expression level/s for gene/s (xi) to (xii) in the sample indicate/s a decreased survival duration relative to the control. References in this paragraph to any single one of the thirteen genes (i) to (xiii) will be relevant only if that gene's expression level was measured.

The invention also provides a method of analyzing a lung tissue sample, comprising a step of measuring the expression level/s in the sample of one or more of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN. As above, the method will typically include a further step of comparing the measured expression level/s to a control level, where the changes (a) to (d) reveal prognostic information about the patient from whom the tissue sample was taken.

The invention also provides a method of analyzing a sample containing RNA transcripts and/or cDNA prepared from a lung cell, comprising a step of measuring the level/s of RNA transcripts and/or cDNA for one or more of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP 1; (xi) MUS81; (xii) VEGFB; and/or (xiii) OPTN. As above, the method will typically include a further step of comparing the measured level/s to a control level, where the changes (a) to (d) reveal prognostic information about the patient from whom the transcripts and/or cDNA was taken.

The invention also provides a metagene comprising at least two (e.g. 2, 3, 4, 5, 6, 7, 8, 9, 10, 11, 12 or all 13) of the following 13 genes: (i) ARPC2; (ii) SDF2; AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN. This metagene (also known as an eigengene) can be used in lung cancer prognosis and diagnosis and represents a group of genes that together exhibit a consistent pattern of expression in relation to an observable phenotype.

The methods of the invention can be used prognostically to predict survival periods for patients, either in combination with current staging or in place of staging.

### Measuring Expression Level/s

Methods of the invention involve measuring the expression level/s of certain gene/s in biological test materials. Genes (i) to (x) have been found to be up-regulated in lung cancer tissue relative to the same tissue from non-cancerous lung, whereas up-regulation of genes (xi) to (xiii) has been associated with the absence of lung cancer. Unless expression of a particular gene is hugely up-regulated or down-regulated (or even absent) then a measured expression level must be compared to a control level in order to determine whether indicates up-regulation, down-regulation or no change.

Various controls can be used to provide a suitable baseline for comparison. Choosing suitable control tissue is routine in the field of diagnostic and prognostic gene expression profiling. For example, a control may be prepared from non-cancerous lung tissue of the same patient as the test material (e.g. obtained earlier in the patient's life at a pre-cancer stage). A control may be prepared from non-cancerous lung tissue of a different patient, in which case levels can optionally be normalized relative to expression levels of a gene that is known not to be down- or up-regulated in lung cancer.

Control levels may be determined in parallel to the determination of levels in the test material. Rather than making a parallel determination in an assay, however, it is normally more convenient to use an absolute control level based on empirical data. For example, the expression levels of a particular gene may be measured in samples taken from a range of patients. If a sample is confirmed by other means (e.g. by histology, etc.) to be non-cancerous then its expression levels can be used to build a picture of baseline expression across the range of patients. This may again involve normalization relative to a reference gene. Usually a population of control patients will be used, to provide a collection of baseline expression levels for patients of different genders, ages, ethnicities, habits (e.g. smokers, non-smokers), etc., so that, if there is variation across the population, the control for test material from a particular patient can be matched to him/her as closely as possible. Thus by analyzing non-cancerous samples from a sufficiently large number of patients it is possible to establish an empirical baseline for any particular gene, which can serve as the control level for comparison according to the invention.

The control level is not necessarily a single value, but could be a range, against which a test value can be compared. For instance, if the expression level of a particular gene is variable across non-cancerous patients, but is always in the range of 50-200 units, an expression level of 500 units in test material indicates up-regulation.

When expression levels in test material are compared to control levels, standard statistical tools can be used to determine whether the levels are the same or different. For example, clinical diagnostics will rarely be based on comparing a single determination for a test material and a control material. Rather, an appropriate number of determinations will be made with an appropriate level of accuracy to give a desired statistical certainty. Expression levels will be measured quantitatively to permit comparison, and enough determinations will be made to ensure that any difference in levels can be assigned a statistical significance to a level of p≦0.05 or better. The number of determinations will vary according to various criteria (e.g. the degree of variation in the baseline, the degree of up-regulation in cancerous tissue, the degree of noise, etc.) but, again, this falls within the normal design capabilities of a person of ordinary skill in this field.

Where a gene is up- or down-regulated then the up- or down-regulation relative to a single baseline level may be defined as a fold difference. Normally it is desirable to use techniques that can indicate a change of at least 1.5-fold up or down e.g. ≧1.75-fold, ≧2-fold, ≧2.5-fold, etc.

In some embodiments, rather than (or in addition to) compare expression levels against a ‘normal’ baseline, they will be compared to levels seen in tumor tissue (i.e. comparison to a positive control). For instance, if the expression level of a particular gene is always at least 500 units in samples from patients with NSCLC, but is lower in normal tissue, it may be easier to make a comparison to this baseline rather than to the lower normal level.

In some embodiments, expression level/s in a sample are compared to expression level/s in one or more positive control samples of lung tumor tissue taken from patient/s with known survival duration/s. The examples show that expression level/s in the metagene have an 83% prognostic accuracy against known survival durations, and so this comparison enables a prediction of the patient's survival duration. Ideally the positive control is a dataset including data obtained from a plurality of patients having known survival durations. With such a dataset then the positive control can provide an average (e.g. median or mean) expression level seen in samples taken from a population of lung cancer patients, and so a comparison can predict whether a patient will survive for a longer or shorter period than the average survival duration of the dataset.

Methods of the invention involve measuring the expression level/s of certain gene/s in biological test material, rather than at levels of polypeptides or other biological molecules. The expression level of a gene is reflected in the quantity of its mRNA transcripts in the test material, and so methods of the invention may involve the measurement of mRNA transcripts. Rather than look at mRNA transcripts directly, however, methods may look at copies and/or complements (whether complete or partial) of such transcripts. Label can conveniently be introduced into such copies/complements during their preparation. Thus the method may, for example, measure cDNA levels (obtained by a step of reverse transcription of the transcripts) or cRNA levels (e.g. obtained by a step of in vitro transcription). During cDNA or cRNA preparation, it is preferred to use methods that substantially retain the relative levels of different transcripts. Methods for purifying RNA transcripts from cells (either for direct analysis, or for preparing cDNA or cRNA), including from lung cancer cells, are well known in the art. A classic RNA isolation protocol is described in reference 10, involving a single-step extraction with an acid guanidine thiocyanate-phenol-chloroform mixture. Commercially available kits such as the TRIZOLT™ total RNA isolation reagent (a mono-phasic solution of phenol and guanidine isothiocyanate, available from Gibco BRL and described in reference 11) may be used, as described in reference 9 for purification of RNA from bronchoscopy samples. Other commercial RNA isolation reagents include RNAqueous™, ToTALLY RNA™, RNAwiz™, Poly(A)Pure™, RNAeasy™, FastTrack™, etc.

Methods for preparing cDNA from cellular RNA transcripts are also well known. The invention may also be used with nucleic acids generated from such cDNAs. For instance, it is known to convert RNA from bronchial epithelial cells into double-stranded cDNA via reverse transcriptase using primers that include a T7 RNA polymerase promoter, and then to perform in vitro transcription on these cDNAs to provide labeled RNA transcripts for analysis [12].

As mentioned above, the invention involves looking at expression levels for at least one of the thirteen genes (i) to (xiii). For any particular patient then the expression levels of a single one of these thirteen genes may give an accurate and adequate prognosis. For a test that is a priori applicable to a broad set of patients, however, it is preferable to measure expression levels for more than one of the genes e.g. 2, 3, 4, 5, 6, 7, 8, 9, 10, 11, 12 or all 13. Analysis of aggregate patterns of gene expression (i.e. metagenes) increases the accuracy (sensitivity and specificity) and confidence for the prognostic result. Multiple genes are preferably analyzed in parallel, thereby providing test results more rapidly. The use of aggregate markers for disease is disclosed in more detail in reference 13. Previous lung cancer metagenes are described in references 9 and 14.

It sometimes happens that expression profiles give ambiguous results e.g. expression of some genes within the metagene indicates disease, whereas expression of other genes indicates no disease. In such a case, if re-testing gives the same result then statistical algorithms can be applied to determine the probability that the patient has a particular metagene score. Statistical algorithms suitable for this purpose are known.

A convenient way of measuring RNA transcript levels for multiple genes in parallel is to use a microarray. Techniques for using microarrays to assess and compare gene expression levels are well known in the art (e.g. see references 15-20) and include appropriate hybridization, detection and data processing protocols. A useful microarray includes multiple nucleic acid probes (typically DNA) that are immobilized on a solid substrate (e.g. a glass support such as a microscope slide, or a membrane) in separate locations such that detectable hybridization can occur between the probes and the transcripts to indicate the amount of each transcript that is present. An array can include multiple probes for each transcript, so as to provide redundancy and permit internal control testing. An array can also include one or more further internal control reagents. The probes on an array can be oligonucleotides (e.g. up to 150 nucleotides) or can be longer (e.g. cDNAs). An array can include probes that focus on the genes of interest herein, or may include probes for a wider range of genes. For example, microarrays for parallel analysis of thousands of human transcripts are available (e.g. Affymetrix™ supplies the HG-U95, HG-U133, and HuGeneFL arrays; Agilent™ supplies the Whole Human Genome Oligo Microarray; Illumina™ supplies the HumanWG-6 and HumanRef-8 Expression BeadChips). Rather than use an array that has expensively been prepared for whole genome analysis, however, it is preferred to use an array that focuses on the genes of interest herein or, as an alternative, on the genes of interest herein and also on genes relevant to other cancers or lung conditions. Many microarray manufacturers will prepare custom arrays for analysis of a specific subset of human transcripts and these custom arrays can rapidly be prepared e.g. by inkjet printing, photolithographic masking, etc.

One way of comparing gene expression in two samples, particularly when using a microarray, is to label a test sample with a first label and a control sample with a second label, where the two labels give distinguishable signals (e.g. a red fluorescence and a green fluorescence). The two samples are then combined and hybridized against the array. If the levels of target in the samples are the same then the two signals will cancel each other out (e.g. a combined red and green signal may be yellow).

Where expression is higher in the test sample then signal from the first label will be more prominent; where expression is higher in the control sample then the second label is more prominent.

Analysis expression levels from an array experiment can be conducted by comparing signal intensities. This can be achieved by generating a ratio matrix of the expression intensities of genes in a test sample versus those in a control sample. A ratio of these expression intensities can be used to provide the fold-change in gene expression between the test and control samples.

Gene expression profiles can be displayed in a number of ways. The most common method is to arrange a ratio matrix into a graphical dendrogram or heatmap where columns indicate samples and rows indicate genes. Data may be arranged so that genes that are expected to have similar expression profiles are grouped together. The expression ratio for each gene can be visualized as a color. For example, down-regulation (relative to a control) may appear in the blue portion of the spectrum whereas up-regulation may be shown using the red portion of the spectrum.

Gene expression profiles may be digitally recorded to facilitate comparison with expression data from other samples.

Another technique for analyzing transcripts is the polymerase chain reaction (PCR), and in particular reverse transcription PCR. Quantitative RT-PCR methods are known in the art and have previously been applied to analyze lung tumors [21] including for measuring expression levels of multiple transcripts in lung cells [22,23] or lung cell lines [24].

Another technique that can be used to study expression levels of multiple genes in lung tissue is serial analysis of gene expression (SAGE) e.g. see reference 25.

Another technique that can be used to study expression levels of multiple genes, with high sensitivity, is the NanoString nCounter gene expression system e.g. see reference 26.

Nucleic acid detection generally involves hybridization between a target (e.g. a transcript or cDNA, as described above) and a probe. Sequences of the 13 genes in the metagene of the invention are known (see below), and so hybridization probes for their detection can readily be designed. Each probe should be substantially specific for its target, to avoid any cross-hybridization and false positives. An alternative to using specific probes is to use specific reagents when deriving materials from transcripts (e.g. during cDNA production, or using target-specific primers during amplification). In both cases specificity can be achieved by hybridization to portions of the targets that are substantially unique within the metagene e.g. hybridization to the polyA tail would not provide specificity. The provision of specific hybridization reagents for 13 unrelated genes is within the ordinary capabilities of a person skilled in the art, and such reagents can be optimized based on experience with them.

Where a target has multiple splice variants and it is desired to detect all of them then it is possible to design a hybridization reagent that recognizes a region common to each variant and/or to use more than one reagent, each of which may recognize one or more variants. Details of splice variants for the 13 different genes in the metagene are disclosed below.

Expression levels of multiple genes can be converted into a ‘metagene score’. For instance, individual expression level changes can be combined using regularized binary regression methods, as described in reference 27. Reference 27 also describes how a metagene score can be converted to a probability scale using binary regression. For the 13 genes in the metagene, individual expression levels may, for instance, be weighted when calculating a metagene score.

Individual expression levels may be weighted as follows when determining aggregate expression patterns for multiple genes within the 13:

| Gene | ARPC2 | SDF2 | AP3D1 | MRPL44 | MYO1E | VEGFB | OPTN |
|---|---|---|---|---|---|---|---|
| Weight | −0.5 | −0.5 | −0.5 | −0.5 | −0.5 | +0.6 | +0.6 |

| Gene | HEBP2 | CSNK1A1 | CLIP1 | MUS81 | ARG2 | SNAP29 |
|---|---|---|---|---|---|---|
| Weight | −0.7 | −0.7 | −0.6 | +0.4 | −0.5 | −0.4 |

In some embodiments each of these weightings may be adjusted by ±0.2 or ±0.1.

For greater precision, the weightings may be as follows, with each of these figures optionally being adjusted by ±0.05, ±0.02: or ±0.01:

| Gene | ARPC2 | SDF2 | AP3D1 | MRPL44 | MYO1E | VEGFB | OPTN |
|---|---|---|---|---|---|---|---|
| Weight | −0.54 | −0.48 | −0.47 | −0.52 | −0.46 | +0.60 | +0.58 |

| Gene | HEBP2 | CSNK1A1 | CLIP1 | MUS81 | ARG2 | SNAP29 |
|---|---|---|---|---|---|---|
| Weight | −0.73 | −0.65 | −0.57 | +0.40 | −0.53 | −0.37 |

The results of expression analysis can be used prognostically to predict survival periods for patients. As shown in FIG. 2B, a high metagene score indicates a short survival period, whereas a low score indicates a longer survival period.

### Samples

The invention involves the analysis of gene expression in lung cells and/or tissues. Lungs include a variety of anatomical types, including the trachea, alveoli, bronchi and bronchioles. The lung contains over 40 different cell types, including epithelial cells, endothelial cells, mesothelial cells, mast cells, clara cells, basement membranes, interstitial cells, lamina propria cells, brush cells, granular cells, pneumocytes, etc. Useful samples for analysis according to the invention may be taken from the bronchial wall, and may thus include a variety of cell types, including but not limited to epithelial cells, glandular cells, myofibroblasts and endothelial cells, as well as mixed in inflammatory cells of different types and amount. Tumor cells in the sample may be derived from, for example, epithelial cells (squamous cell cancer) or glandular cells (adenocarcinomas). One useful aspect of the present invention is that it has been demonstrated to give useful results even in samples that contain differing proportions of mixed cell types, with a high prognostic accuracy being maintained even with varying degrees of tumor cell content. Thus the methods avoid the need to isolate tumor cells from biopsies beforehand, thereby avoiding the need for techniques such as laser capture microdissection that would not easily be added to current cancer diagnosis workflows.

Lung tissue samples for use with the invention will typically be obtained by bronchoscopy. The bronchoscope may be rigid, but is preferably flexible. Samples that are obtained by bronchoscopy include biopsies, fluids (bronchoalveolar lavage), or endobronchial brushing samples. Samples obtained by bronchial brushes typically contain cells from only superficial regions of the bronchial wall, and these cells often show signs of apoptosis and decreased viability. Rather than use brushing samples, therefore, the invention is particularly useful with bronchoscopic biopsies. An advantage of bronchoscopy for obtaining samples is that it is safe, almost non-invasive (particularly with a flexible bronchoscope), and applicable to patients with early as well as advanced disease [28]. Moreover, it already represents a cornerstone of the standard clinical work-up of patients with suspected lung cancer [29]. Thus the use of bronchial biopsies is applicable to almost every patient and can easily be implemented in standard clinical work-up [30], thereby requiring minimal modification to existing protocols. Moreover, in contrast to brushing samples, bronchial biopsies can be used to assess whether tumor cells have penetrated the lamina propria as a proof of invasivity—an important cornerstone of diagnosing lung cancer.

Ideally, at least 1% (e.g. ≧5%, ≧10%, ≧15%, ≧20%, ≧25% or more) of the cells in a sample analyzed by the methods of the invention are tumor cells.

After a sample is removed from a patient then, if it cannot be processed immediately, it can be treated to stabilize its RNA content and prevent degradation. This may involve freezing, but room temperature protocols are also known. For example, the RNAlater™ regent from Ambion™ is an aqueous, non-toxic tissue storage reagent that rapidly permeates tissues to stabilize and protect cellular RNA. Tissue pieces can be harvested and submerged in RNAlater™ for storage without jeopardizing the quality or quantity of RNA obtained after subsequent RNA isolation. The RNAlater product is described in more detail in reference 31 and may contain ammonium sulfate, sodium citrate and EDTA in aqueous solution (e.g. 25 mM sodium citrate, 10 mM EDTA, 70 g ammonium sulfate per 100 ml solution, pH 5.2).

Although the invention may be useful with a variety of mammals, it is mainly intended for humans.

### Lung Cancers

The invention analyzes gene expression in lung cells to provide information that is useful in the diagnosis and/or prognosis of lung cancers. The most common lung cancers are small cell lung cancer (SCLC) and non-small cell lung cancer (NSCLC), which are treated differently. Other lung cancers include carcinoid tumors and large cell neuroendocrine carcinoma. The invention is particularly useful for the prognosis of NSCLC.

NSCLC is the most common type of lung cancer and has three sub-types that differ in size and shape: squamous cell carcinomas, which tend to be found in the middle of the lungs, near a bronchus; adenocarcinomas, which are usually found in the outer part of the lung; and large-cell (undifferentiated) carcinomas, including spindle cell carcinomas and large cell neuroendocrine carcinomas, which can start in any part of the lung and usually grow and spread quickly. Sometimes tumors may fall into two sub-types e.g. adenosquamous carcinoma.

NSCLC can be staged using the AJCC or UICC system, with stages 0, I, II, III or IV. Stages I, II and III may be further divided into A and B. Staging is currently used to predict survival periods for patients, but the metagene of the invention is at least equivalent to UICC-stages for these predictions.

Although the three sub-types are histo-morphologically distinct, sub-typing is not of predictive or prognostic relevance and so does not currently translate to differences in treatment i.e. the different histological subtypes of NSCLC are currently treated according to the same protocols.

### ARPC2

ARPC2 is one of the 13 genes that can be analyzed according to the invention. It encodes the actin-related protein 2/3 complex, subunit 2, 34 kDa. It has also been referred to as ARC34, PRO2446, p34-Arc and PNAS-139. The HGNC (HUGO Gene Nomenclature Committee, which aims to give unique and meaningful names to every human gene) has given this gene unique ID HGNC:705.

ARPC2 is one of seven subunits of the human Arp2/3 protein complex. The Arp2/3 protein complex has been implicated in the control of actin polymerization in cells and has been conserved through evolution. 12 splice variants are included in the Alternative Splicing Database (ASD) [32], and two alternatively spliced variants have been characterized in detail. The NCBI Reference Sequences (RefSeq) for ARPC2 are NM_{—}005731 (GI:23238209; SEQ ID NO: 1) and NM_{—}152862 (GI:23238210; SEQ ID NO: 2).

Up-regulated expression of ARPC2 has herein been associated with a poor prognosis. Several previous studies suggested that ARPC2 together with Wiskott-Aldrich syndrome family verproline-homologous protein 2 (WAVE2) are implicated in the formation of protrusion structures by actin polymerization which result in the initiation of cellular migration [33]. Co-expression of these two proteins has been shown to predict poor outcome in AC of the lung.

Probes for ARPC2 are present in Affymetrix arrays U95 and U133. There are currently 8 TaqMan™ PCR assays for ARPC2 available from ABI, with amplicon lengths ranging from 62 bp to 132 bp. These assay products can be used with the present invention. More generally, expression of ARPC2 transcripts can be detected by the use of nucleic acids that hybridize to SEQ ID NO: 1 or SEQ ID NO: 2 (or to the complements thereof) or a splice variant thereof.

### SDF2

SDF2 is one of the 13 genes that can be analyzed according to the invention. It encodes the stromal cell-derived factor 2. The HGNC unique ID for SDF2 is HGNC:10675. The protein encoded by this gene is believed to be a secretory protein and it has regions of similarity to hydrophilic segments of yeast mannosyltransferases. Its expression is ubiquitous and the gene appears to be relatively conserved among mammals Seven splice variants are included in the ASD. The RefSeq for SDF2 is NM_{—}006923 (GI: 14141194; SEQ ID NO: 3).

Up-regulated expression of SDF2 has herein been associated with a poor prognosis.

Probes for SDF2 are present in Affymetrix arrays U95 and U133. There are currently 3 TaqMan™ PCR assays for ARPC2, with amplicon lengths ranging from 63 bp to 89 bp. These assay products can be used with the present invention. More generally, expression of SDF2 transcripts can be detected by the use of nucleic acids that hybridize to SEQ ID NO: 3 (or to the complement thereof) or a splice variant thereof.

### AP3D1

AP3D1 is one of the 13 genes that can be analyzed according to the invention. It encodes adaptor-related protein complex 3, delta 1 subunit. It has also been referred to as ADTD and hBLVR. The HGNC unique ID for AP3D1 is HGNC:568. AP3D1 is a subunit of the AP3 adaptor-like complex, which is not associated with clathrin. The AP3D1 subunit is implicated in intracellular biogenesis and trafficking of pigment granules and possibly platelet dense granules and neurotransmitter vesicles. 13 splice variants are included in the ASD. The RefSeqs for two isoforms of AP3D1 are NM_{—}001077523 (GI:117553583; SEQ ID NO: 4) and NM_{—}003938 (GI:117553579; SEQ ID NO: 5).

Up-regulated expression of AP3D1 has herein been associated with a poor prognosis.

Probes for AP3D1 are present in Affymetrix arrays U95 and U133. There are currently 28 TaqMan™ PCR assays for AP3D1, with amplicon lengths ranging from 56 bp to 106 bp. These assay products can be used with the present invention. More generally, expression of AP3D1 transcripts can be detected by the use of nucleic acids that hybridize to SEQ ID NO: 4 or SEQ ID NO: 5 (or to the complements thereof) or a splice variant thereof.

### MRPL44

MRPL44 is one of the 13 genes that can be analyzed according to the invention. It encodes 39S mitochondrial ribosomal protein L44. It has also been referred to as FLJ12701 and FLJ13990. The HGNC unique ID for MRPL44 is HGNC:16650. The RefSeq for MRPL44 is NM_{—}022915 (GI: 21735610; SEQ ID NO: 6).

Up-regulated expression of MRPL44 has herein been associated with a poor prognosis.

Probes for MRPL44 are present in Affymetrix arrays U95 and U133. There are currently 3 TaqMan™ PCR assays for MRPL44, with amplicon lengths ranging from 69 bp to 98 bp. These assay products can be used with the present invention. More generally, expression of MRPL44 transcripts can be detected by the use of nucleic acids that hybridize to SEQ ID NO: 6 (or to the complement thereof) or a splice variant thereof.

### MYO1E

MYO1E is one of the 13 genes that can be analyzed according to the invention. It encodes myosin IE. It has also been referred to as MYO1C, HuncM-IC and MGC104638. The HGNC unique ID for MYO1E is HGNC:7599. 12 splice variants are included in the ASD. The RefSeq for MYO1E is NM_{—}004998 (GI: 55956915; SEQ ID NO: 7).

Up-regulated expression of MYO1E has herein been associated with a poor prognosis.

Probes for MYO1E are present in Affymetrix arrays U95 and U133. There are currently 23 TaqMan™ PCR assays for MYO1E, with amplicon lengths ranging from 60 bp to 157 bp. These assay products can be used with the present invention. More generally, expression of MYO1E transcripts can be detected by the use of nucleic acids that hybridize to SEQ ID NO: 7 (or to the complement thereof) or a splice variant thereof.

### ARG2

ARG2 is one of the 13 genes that can be analyzed according to the invention. It encodes arginase, type II. The HGNC unique ID for ARG2 is HGNC:664. Arginase catalyzes the hydrolysis of arginine to ornithine and urea, and the type II isoform is located in the mitochondria and expressed in extra-hepatic tissues. The physiologic role of this isoform is poorly understood, but it is thought to play a role in nitric oxide and polyamine metabolism. Transcript variants of the type II gene resulting from the use of alternative polyadenylation sites have been described, and 4 splice variants are included in the ASD. The RefSeq for ARG2 is NM_{—}001172 (GI: 52426739; SEQ ID NO: 8).

Up-regulated expression of ARG2 has herein been associated with a poor prognosis. This matches a previous study [34] that considered arginases as poor markers of prognosis in human NSCLC.

Probes for ARG2 are present in Affymetrix arrays U95 and U133. There are currently 7 TaqMan™ PCR assays for ARG2, with amplicon lengths ranging from 61 bp to 141 bp. These assay products can be used with the present invention. More generally, expression of ARG2 transcripts can be detected by the use of nucleic acids that hybridize to SEQ ID NO: 8 (or to the complement thereof) or a splice variant thereof.

### SNAP29

SNAP29 is one of the 13 genes that can be analyzed according to the invention. It encodes synaptosomal-associated protein, 2910a. It has also been referred to as CEDNIK and FLJ21051. The HGNC unique ID for SNAP29 is HGNC:11133. SNAP29 is a member of the SNAP25 gene family and encodes a protein involved in multiple membrane trafficking steps. The protein encoded by SNAP29 binds tightly to multiple syntaxins and is localized to intracellular membrane structures rather than to the plasma membrane. While the protein is mostly membrane-bound, a significant fraction of it is found free in the cytoplasm. Use of multiple polyadenylation sites has been noted for this gene. The RefSeq for SNAP29 is NM_{—}004782 (GI: 18765736; SEQ ID NO: 9).

Up-regulated expression of SNAP29 has herein been associated with a poor prognosis.

Probes for SNAP29 are present in Affymetrix arrays U95 and U133. There are currently 3 TaqMan™ PCR assays for SNAP29, with amplicon lengths ranging from 75 bp to 98 bp. These assay products can be used with the present invention. More generally, expression of SNAP29 transcripts can be detected by the use of nucleic acids that hybridize to SEQ ID NO: 9 (or to the complement thereof) or a splice variant thereof.

### HEBP2

HEBP2 is one of the 13 genes that can be analyzed according to the invention. It encodes heme binding protein 2. It has also been referred to as PP23, SOUL, C6orf34, C60RF34B, KIAA1244 and RP3-422G23.1. The HGNC unique ID for HEBP2 is HGNC:15716. 3 splice variants are included in the ASD. The RefSeq for HEBP2 is NM_{—}014320 (GI: 41393567; SEQ ID NO: 10).

Up-regulated expression of HEBP2 has herein been associated with a poor prognosis.

Probes for HEBP2 are present in Affymetrix arrays U95 and U133. There are currently 3 TaqMan™ PCR assays for HEBP2, with amplicon lengths ranging from 61 bp to 79 bp. These assay products can be used with the present invention. More generally, expression of HEBP2 transcripts can be detected by the use of nucleic acids that hybridize to SEQ ID NO: 10 (or to the complement thereof) or a splice variant thereof.

### CSNK1A1

CSNK1A1 is one of the 13 genes that can be analyzed according to the invention. It encodes casein kinase 1, alpha 1. It has also been referred to as CK1, HLCDGP1 and PRO2975. The HGNC unique ID for CSNK1A1 is HGNC:2451. 8 splice variants are included in the ASD. The RefSeq for CSNK1A1 is NM_{—}001025105 (GI: 68303574; SEQ ID NO: 11).

Up-regulated expression of CSNK1A1 has herein been associated with a poor prognosis.

Probes for CSNK1A1 are present in Affymetrix arrays U95 and U133. There are currently 5 TaqMan™ PCR assays for CSNK1A1, with amplicon lengths ranging from 72 bp to 134 bp. These assay products can be used with the present invention. More generally, expression of CSNK1A1 transcripts can be detected by the use of nucleic acids that hybridize to SEQ ID NO: 11 (or to the complement thereof) or a splice variant thereof.

### CLIP1

CLIP1 is one of the 13 genes that can be analyzed according to the invention. It encodes the CAP-GLY domain containing linker protein 1. It has also been referred to as RSN, CLIP, CYLN1, CLIP170 and MGC131604. The HGNC unique ID for CLIP1 is HGNC:10461. 9 splice variants are included in the ASD. The RefSeq for CLIP1 is NM_{—}002956 (GI: 38016917; SEQ ID NO: 12).

Up-regulated expression of CLIP1 has herein been associated with a poor prognosis.

Probes for CLIP1 are present in Affymetrix arrays U95 and U133. There are currently 23 TaqMan™ PCR assays for CLIP1, with amplicon lengths ranging from 65 bp to 154 bp. These assay products can be used with the present invention. More generally, expression of CLIP1 transcripts can be detected by the use of nucleic acids that hybridize to SEQ ID NO: 12 (or to the complement thereof) or a splice variant thereof.

### MUS81

MUS81 is one of the 13 genes that can be analyzed according to the invention. It encodes the homolog of S. cerevisiae MUS81 protein. It has also been referred to as FLJ21012 and FLJ44872. The HGNC unique ID for MUS81 is HGNC:29814. 10 splice variants are included in the ASD. The RefSeq for MUS81 is NM_{—}025128 (GI: 156151412; SEQ ID NO: 13).

Up-regulated expression of MUS81 has herein been associated with a good prognosis.

Probes for MUS81 are present in Affymetrix arrays U95 and U133. There are currently 12 TaqMan™ PCR assays for MUS81, with amplicon lengths ranging from 63 bp to 127 bp. These assay products can be used with the present invention. More generally, expression of MUS81 transcripts can be detected by the use of nucleic acids that hybridize to SEQ ID NO: 13 (or to the complement thereof) or a splice variant thereof.

### VEGFB

VEGFB is one of the 13 genes that can be analyzed according to the invention. It encodes vascular endothelial growth factor B. It has also been referred to as VRF and VEGFL. The HGNC unique ID for VEGFB is HGNC:12681. Two splice variants are included in the ASD. The RefSeq for VEGFB is NM_{—}003377 (GI: 39725673; SEQ ID NO: 14).

Up-regulated expression of VEGFB has herein been associated with a good prognosis.

Probes for VEGFB are present in Affymetrix arrays U95 and U133. There are currently 4 TaqMan™ PCR assays for VEGFB, with amplicon lengths ranging from 52 bp to 86 bp. These assay products can be used with the present invention. More generally, expression of VEGFB transcripts can be detected by the use of nucleic acids that hybridize to SEQ ID NO: 14 (or to the complement thereof) or a splice variant thereof.

### OPTN

OPTN is one of the 13 genes that can be analyzed according to the invention. It encodes optineurin. It has also been referred to as NRP, FIP2, HIP7, HYPL, GLC1E and TFIIIA-INTP. The HGNC unique ID for OPTN is HGNC:17142. Optineurin is a coiled-coil containing that interacts with adenovirus E3-14.7K protein and may utilize TNF-α or Fas-ligand pathways to mediate apoptosis, inflammation or vasoconstriction. Optineurin may also function in cellular morphogenesis and membrane trafficking, vesicle trafficking, and transcription activation through its interactions with the RAB8, huntingtin, and transcription factor IIIA proteins. Alternative splicing results in multiple transcript variants, with some encoding the same protein, and 12 splice variants are included in the ASD. The four RefSeqs for OPTN are NM_{—}001008211 (GI: 56549106; SEQ ID NO: 15), NM_{—}001008212 (GI: 56549108; SEQ ID NO: 16), NM_{—}001008213 (GI: 56549110; SEQ ID NO: 17) and NM_{—}021980 (GI: 56550112; SEQ ID NO: 18).

Up-regulated expression of OPTN has herein been associated with a good prognosis.

Probes for OPTN are present in Affymetrix arrays U95 and U133. There are currently 19 TaqMan™ PCR assays for OPTN, with amplicon lengths ranging from 55 bp to 137 bp. These assay products can be used with the present invention. More generally, expression of OPTN transcripts can be detected by the use of nucleic acids that hybridize to SEQ ID NO: 15 or SEQ ID NO: 16 or SEQ ID NO: 17 or SEQ ID NO: 18 (or to the complements thereof) or a splice variant thereof.

### Patient Treatment

The invention describes methods of prognosis of a lung cancer in a patient, in which gene expression in lung cells and/or tissues are analyzed. If a sample shows up-regulation of genes (i) to (x) then there is a strong likelihood of poor survival in the patient. In the event of such a result, therefore, the invention may then include one or more of the following steps: informing the patient that they are likely to have lung cancer with a poor survival duration; confirmatory histological examination of lung tissue; and/or treating the patient by a lung cancer therapy.

Typical initial NSCLC combination chemotherapies include administration of: paclitaxel and carboplatin; gemcitabine and cisplatin; gemcitabine and carboplatin; vinorelbine and cisplatin; or docetaxel and cisplatin. Thus a method of the invention may, after a positive result, involve administration of one or more or paclitaxel, carboplatin, gemcitabine, cisplatin, vinorelbine and/or docetaxel.

### Products

The invention provides a device comprising immobilized nucleic acid probes (typically DNA) for detecting transcripts from two or more (e.g. 2, 3, 4, 5, 6, 7, 8, 9, 10, 11, 12 or all 13) of the following 13 human genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.

The device may include immobilized nucleic acid probes for more than just these 13 genes, but preferably it includes probes for fewer than 5000 genes (e.g. <4000, <3000, <2000, <1000, <500, <250, <100, <50, <25, etc.)

The device can use any suitable support material e.g. glass, plastic, nylon, etc. The probes may be oligonucleotides (e.g. up to 150 nucleotides) or longer (e.g. cDNAs). The probes may be synthesized and then attached to the support, or they may be built in situ on the support (e.g. by inkjet printing as in Agilent™ array products, photolithographic masking as in Affymetrix™ array products, etc.). Probes may be attached to bead supports, which are then deposited onto a surface, as in Illumina™ array products.

The invention also provides a kit for conducting a method of the invention, comprising primers and/or probes for amplifying and/or detecting transcripts from two or more (e.g. 2, 3, 4, 5, 6, 7, 8, 9, 10, 11, 12 or all 13) of the following 13 human genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN. The primers may be suitable for PCR, SDA, SSSR, LCR, TMA, NASBA, etc.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

“GI” numbering is used above. A GI number, or “GenInfo Identifier”, is a series of digits assigned consecutively to each sequence record processed by NCBI when sequences are added to its databases. The GI number bears no resemblance to the accession number of the sequence record.

When a sequence is updated (e.g. for correction, or to add more annotation or information) then it receives a new GI number. Thus the sequence associated with a given GI number is never changed.

### BRIEF DESCRIPTION OF DRAWINGS

FIG. 1 shows a biplot representation of between-group analysis.

FIG. 2 shows graphs relating to survival analyses.

### MODES FOR CARRYING OUT THE INVENTION

Bronchoscopic biopsy samples were collected from 56 patients undergoing flexible video-bronchoscopy for suspicion of lung cancer. The samples were immediately stored in RNAlater™ (Ambion) and then frozen at −20° C. within 1 hour.

For histopathological diagnosis the biopsies were fixed in 4% buffered formalin, paraffin-embedded, cut at 4 μm and stained with haematoxylin and eosin, alcian blue periodic acid shift and elastica van Gieson according to routine procedures. This histopathology was combined with cytology, mediastinoscopy, or CT-guided biopsy to give a positive or negative cancer diagnosis. Thus the patients were diagnosed as suffering either from NSCLC (41 patients, with appropriate sub-classification into adenocarcinoma or squamous cell carcinoma where possible, and also with staging by UICC criteria) or merely from chronic inflammatory lung disease (15 patients, providing a control group). The NSCLC and control groups were matched for age and gender.

With this diagnosis in place, a study of gene expression between the NSCLC and control groups was performed. RNA was extracted from the samples and amplified by in vitro transcription using the Ambion Ally MessageAmp Kit™ to produce cRNA. The amplified transcripts contain aminoallyl UTPs to which Cy5 dyes were attached, and then hybridized to Novachip™ microarrays.

The hybridization results were log-transformed, centered and normalized by scaling the intensity distribution using the 75% trimmed mean, and variance was stabilized by logarithmic transformation. Technical batch effects were adjusted using Partek™ batch removal software. NSCLC class comparison was performed using unsupervised hierarchical clustering [35] and supervised between group analysis (BGA) [36]. For maximum specificity in the supervised class comparison the analysis was restricted to samples in which a pathologist had detected tumor cells. Class prediction accuracy was assessed using a genetic algorithm (including a 2-level crossvalidation) combined with the nearest centroid classification method (implemented in the ‘Galgo’ R package [37]).

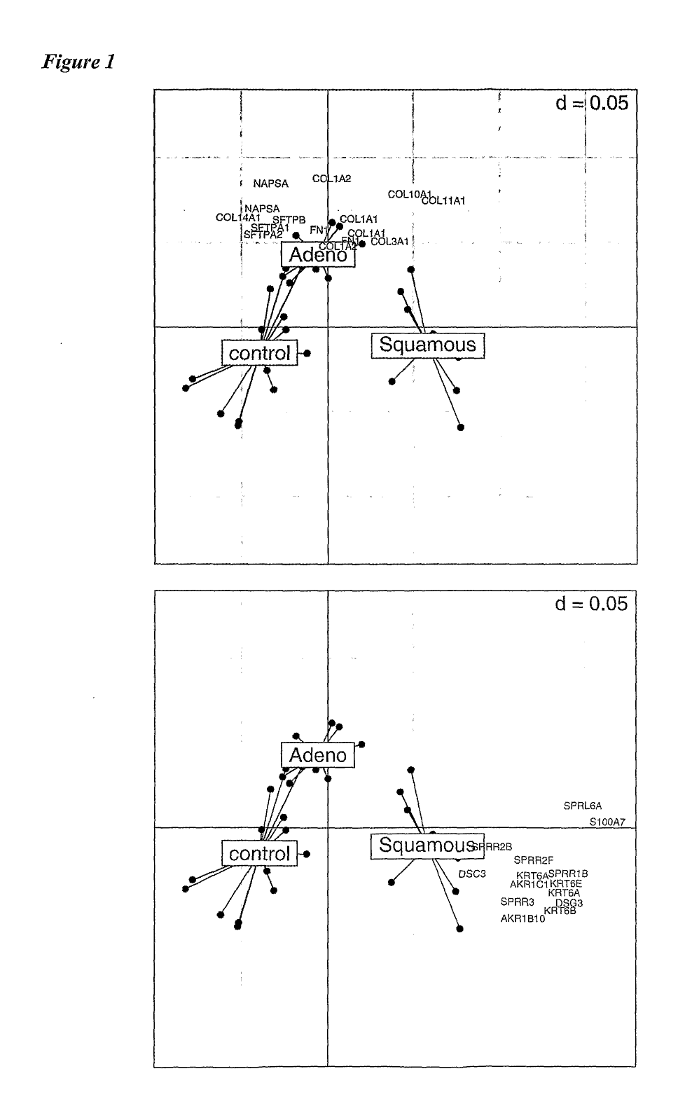

The BGA identified various genes that could discriminate between phenotypes. FIG. 1 shows a biplot representation of between-group analysis and significantly discriminates the three groups of patients (P=0.001). The main effect supported by the first discriminating axis (76%) separates SCC from C. The second BGA axis separates AC from the two other groups. The most discriminating genes have the highest absolute scores on the BGA axes. FIG. 1 includes examples of genes strongly expressed in SCC (top panel) and AC (bottom panel).

67 of the 100 most discriminating genes were already described in the literature as being associated with lung cancer. SCC typically exhibited an up-regulation of keratin genes, genes associated with epithelial development such as Ca²⁺-binding proteins, small proline-rich proteins, desmosomal proteins, and antioxidant proteins such as aldo-keto reductases. AC showed increased transcriptional levels of markers routinely used for the diagnosis of lung adenocarcinomas such as surfactant proteins and aspartic proteinase (Napsin A). The 45 most informative genes identified by genetic algorithm were used for phenotype predictions. Overall sensitivity and specificity was respectively 0.80 and 0.89.

Survival analysis was carried out by applying univariate Cox proportional-hazard regression and supervised principal component analysis [38]. A metagene based upon a linear combination of the most discriminating genes was built according to the procedure described in reference 38. Based on the median of the metagene scores, a binary score (low/high risk) was built and the survival results were displayed using Kaplan-Meyer curves. The survival analysis was performed for all 41 NSCLC patients. The cancer stage was the only highly significant clinical predictor of survival (P<0.001). Cox proportional-hazards regression models including stage as co-variable were fitted gene-by-gene. Genes were ranked according to their hazard ratio. A metagene including 44 genes gave the most accurate prediction of survival (P<0.001). The metagene had 34 risk genes and 10 protective genes.

Of these 44 genes, 13 (10 risk genes and 3 protective genes) could be validated as being significantly associated with survival using four recently-published independent lung cancer data sets [1, 3, 5, 39] that used 3 different gene expression platforms, and included patients from different continents, ethnicities and races. The 10 risk genes were (i) ARPC2; (ii) SDF2; (iii) AP3D1; (iv) MRPL44; (v) MYO1E; (vi) ARG2; (vii) SNAP29; (viii) HEBP2; (ix) CSNK1A1; and (x) CLIP1. The 3 protective genes were (xi) MUS81; (xii) VEGFB; and (xiii) OPTN. With these 13 genes, a metagene was built and tested.

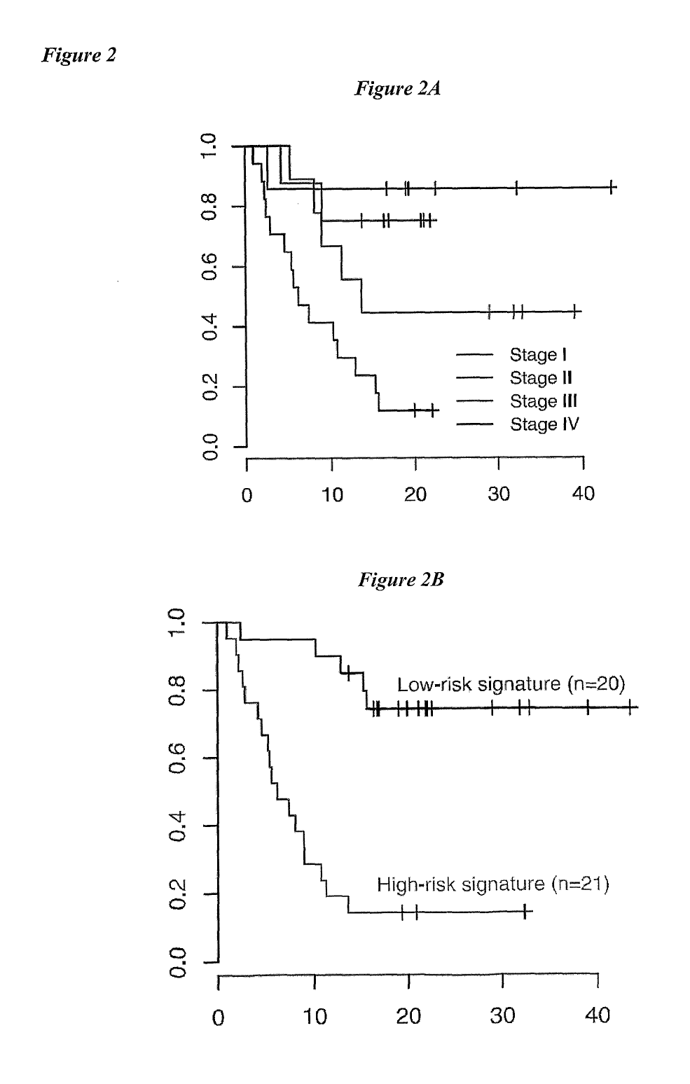

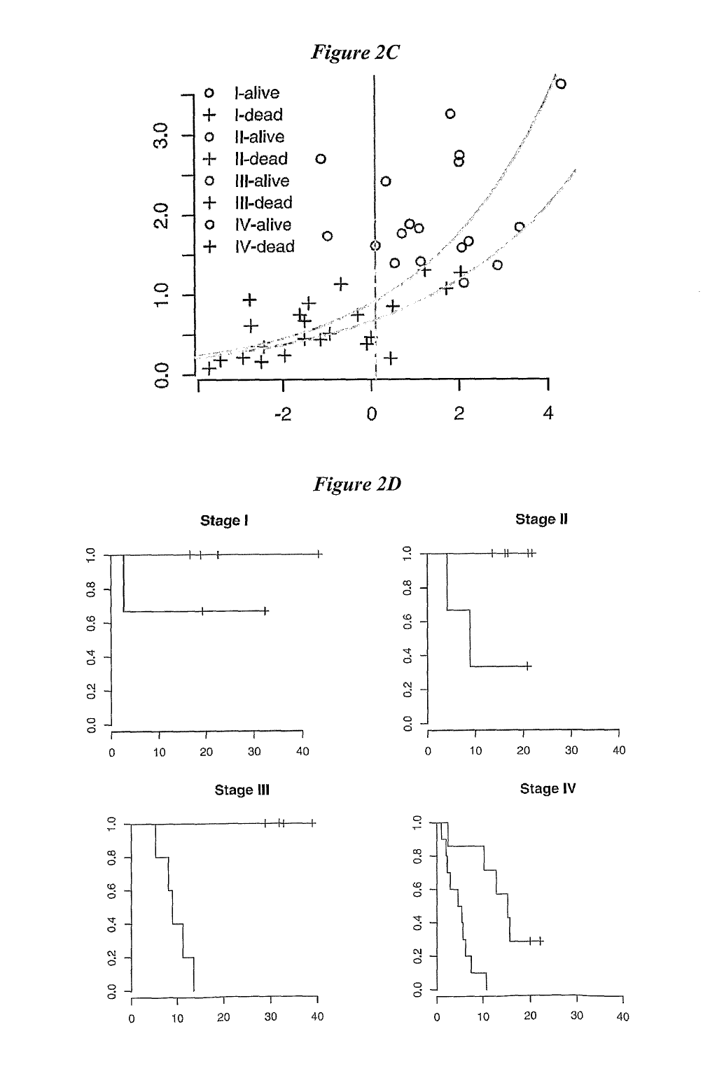

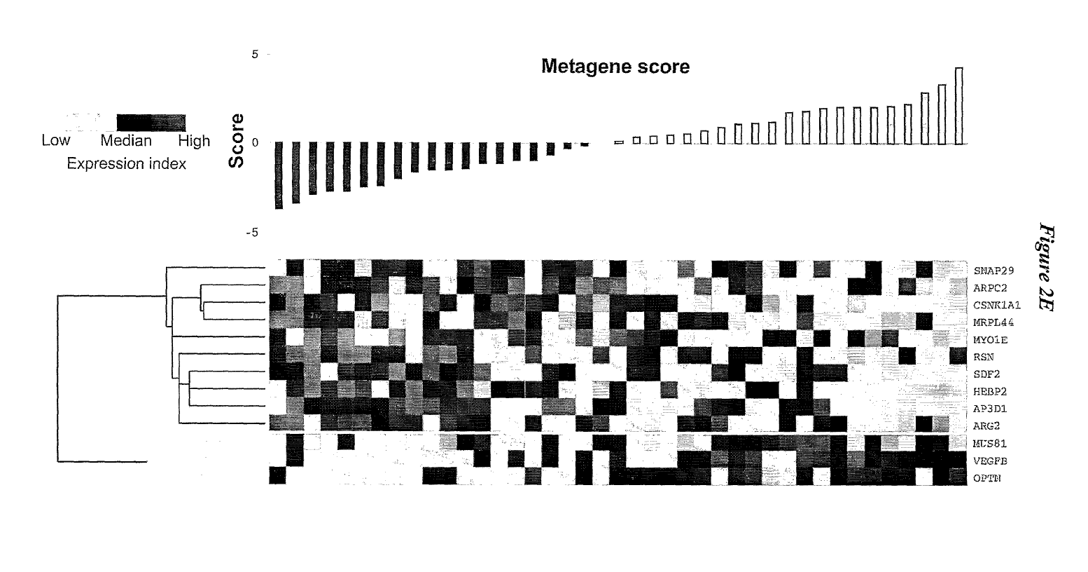

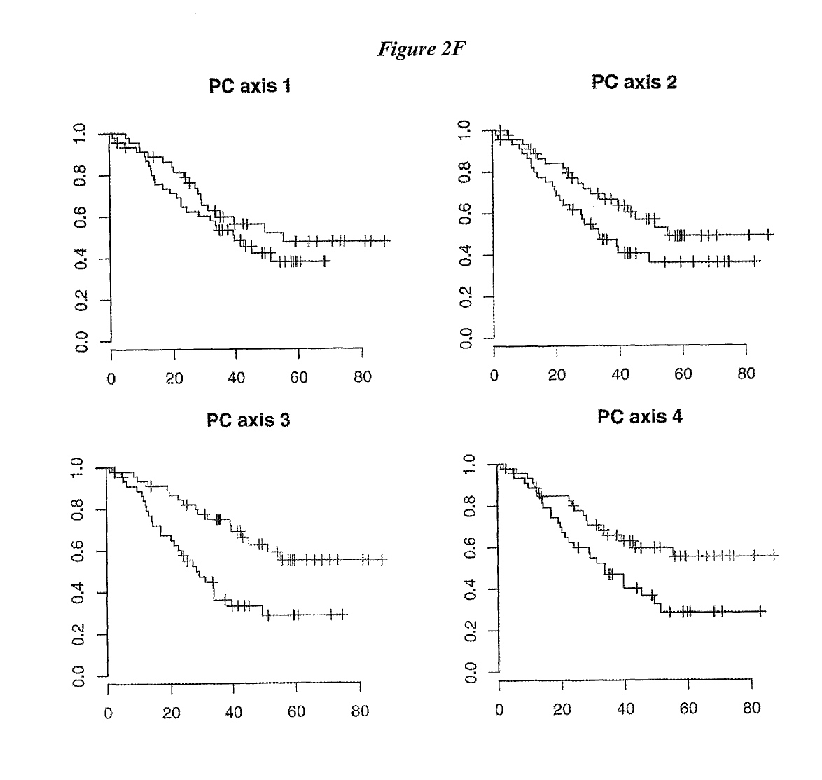

FIG. 2A shows the Kaplan-Meier estimates of survival according to the 4 UICC-stages I to IV. FIG. 2B shows the Kaplan-Meier estimates based on the 13-gene metagene. The metagene gives independent prognostic information complimentary to UICC-stages (P<0.001). FIG. 2C shows survival (crosses) and follow-up (circles; alive patients) as a function of the metagene scores. FIG. 2D shows the Kaplan-Meier estimates of survival for the indicated UICC-stages after subdivision into low- and high-risk according to the metagene scores. When combining both the UICC-stage and the metagene, a significant gain of fit was obtained (P<0.001). The metagene score was particularly good in identifying patients with a survival of less than 1 year, independently of the UICC-tumor stage (sensitivity/specificity 0.78/0.89).

With these 13 genes, a metagene score was calculated for each patient (FIG. 2E). Each column in FIG. 2E represents a single patient, and the magnitude of the metagene score was in relation to survival, with a low score is associated with chance of short survival.

Of the 13 genes, 3 appeared to be protective: MUS81, OPTN and VEGFB. The relevance of VEGFB was further validated using immunohistochemistry on tissue microarrays [40] with tumor samples from 508 fully annotated patients. For these 508 patients a primary lung carcinoma had been analyzed and there was adequate follow-up information for suitable evaluation. Average patient age within the 508 patients was 63 years. For each patient it was judged whether or not the lung tumor was the cause of death. As study endpoints, survival time (independent of cause of death), survival time until tumor-related death (tumor-specific survival time) were used. For all tumors, histological sections were re-evaluated. Tumor type was defined and tumor grading as low-grade or high-grade malignant was performed. Well-differentiated squamous and adenocarinomas, as well as bronchoalveolar carcinomas, were defined as low-grade malignant; all others as high-grade malignant. Tumor stage and degree of differentiation were judged according to UICC and WHO criteria. Additional data such as pT stage and pN stage were retrieved from the pathology reports.

The histopathological distribution of tumors was as follows:

| Stage | | pT1 | pT2 | pT3 | pT4 | Alle |
|---|---|---|---|---|---|---|
| Total | N | 116 | 317 | 64 | 9 | 506 |
| Number highly malignant | N | 79 | 230 | 48 | 8 | 365 |
| (p = 0.4) | % | 68.1 | 72.3 | 76.6 | 88.9 | 72.1 |
| Number pN+ | N | 37 | 133 | 38 | 5 | 213 |
| (p = 0.004) | % | 31.9 | 42.0 | 59.4 | 55.6 | 42.1 |

For 487 (95.9%) of the 508 patients a tumor specific survival time could be calculated. 293 patients died after a mean follow up time of 28.8 months (0-171.0 months). 21 patients had to be excluded from the calculation due to unclear circumstances of death. A relapse occurred for 211 (41.5%) of 508 patients after a mean of 18.0 months with a mean observation time of 39.9 months. Relapses were distant metastases in 115 cases (56.8%), loco-regional in 56 cases (27.5%) and for 32 patients (15.7%) loco-regional in combination with distant metastases. Of 309 patients with available smoking history, 231 had stopped smoking (74.8%) and only 29 (9.4%) had never smoked. Smokers had smoked between 1 and 140 pack years (average of 44.4 pack years).

pT and pN stage were tightly (and independently) correlated with patient prognosis as expected (p=0.0005, p<0.0001). The degree of differentiation has also influence on prognosis (p=0.0042) but is not an independent prognostic factor (p=0.15) in multivariate analysis including pT and pN. Small cell carcinomas had worse prognosis than non-small cell carcinomas but were underrepresented in our population (N=7), preventing a reliable statistical analysis.

Based on the 508 patients, the protective property of VEGFB was confirmed, and patients with significant expression of VEGFB have a significantly higher survival (P=0.038). This result contrasts with the association of VEGFB with negative prognosis reported in references 41 and 42 but was confirmed at the protein level by using tissue microarrays on a cohort of 508 patients with NSCLC.

The subset of 13 genes was tested on the Bild data set [39]. A linear combination of these genes using supervised principle component analysis (PCA) yielded to a set of metagenes. The second, third and fourth PC were significant predictors of survival (FIG. 2F). The 4 panels in FIG. 2F correspond to Kaplan-Meier curves of survival modeled by the 4 dominant metagenes obtained after supervised principal component analysis. The patient categorization was based on the median score of the metagenes. Thus, in addition to the first PC containing variations unrelated with survival, the inclusion of the second PC was required to reliably predict survival (likelihood ratio test P=0.007).

By using a nearest centroids classifier after feature selection from a genetic algorithm we could reach a sensitivity of 0.77 and a specificity of 0.91 for the prediction of individuals from the control group.

The impact of tumor cell content in the biopsies was assessed both in terms of diagnostic and prognostic accuracy. The estimation of the proportion of tumor cells was done by two independent pathologists on either a cut half of the biopsy, which was used for the gene expression profiling, or a bronchoscopic biopsy taken from the same area during the same bronchoscopy. The prediction accuracy was dependent on the presence and proportion of tumor cells present in the biopsies (Kruskal-Wallis test: P<0.001). The median diagnostic accuracy was 39% when no tumor cells were found in the biopsies, whereas it was 87% in case of at least 1% visible tumor cells. On the other hand, the prognostic accuracy of the metagene—as measured by the absolute value of the individual residual error—did not significantly differ with varying degree of tumor cell content (Kruskal-Wallis test: P=0.79). Thus the tissue surrounding the tumor seems to carry sufficient and significant prognostic gene expression signals, such that biopsies with ≧1% tumor cells can, using modern statistical tools, provide relevant and specific diagnostic/prognostic gene expression signatures without the need for labor-intensive cell purification methods.

Thus analysis of gene expression in bronchoscopic biopsies obtained during initial diagnostic work for NSCLC is feasible and reveals reliable tumor-specific and prognostic gene signals. The proposed approach results in diagnostic and prognostic information complimentary to histopathologic examination and UICC-staging. Before this work all gene expression microarray studies investigating outcome of patients with lung cancer have used tumor biopsies from surgical resections, which limits the application to operable and early stages. The sensitivity and specificity to identify the correct diagnosis was 80 and 90% respectively. A proportion of tumor cells within the biopsies of ≧1% was necessary for a reliable classification. 67% of genes used to discriminate between the different phenotypes have already been described in the literature as being associated with lung cancer, which confirms the biological adequacy of the method even though the biopsies contained differing proportions of mixed cell types. With the aid of a metagene including 44 genes it was possible to accurately predict survival of patients with NSCLC. Using four independent data sets, 13 genes were validated as showing a significant association with the survival of NSCLC patients. Among them, VEGFB gene was validated on a protein level using tissue microarray technology. The proposed metagene score is at least as equivalent to the UICC stages for prediction of survival and was particularly efficient to identify patients with a survival of less than 1 year independently of the UICC-tumor stage.

It will be understood that the invention has been described by way of example only and modifications may be made whilst remaining within the scope and spirit of the invention.

### REFERENCES

[1] Bhattacharjee et al. Classification of human lung carcinomas by mRNA expression profiling reveals distinct adenocarcinoma subclasses. Proc Natl Acad Sci USA 2001; 98:13790-5
[2] Garber M E, Troyanskaya O G, Schluens K et al. Diversity of gene expression in adenocarcinoma of the lung. Proc Natl Acad Sci USA 2001; 98:13784-9
[3] Beer D G, Kardia S L, Huang C C et al. Gene-expression profiles predict survival of patients with lung adenocarcinoma. Nat Med 2002; 8:816-24
[4] Raponi M, Zhang Y, Yu J et al. Gene expression signatures for predicting prognosis of squamous cell and adenocarcinomas of the lung. Cancer Res 2006; 66:7466-72.
[5] Tomida S, Koshikawa K, Yatabe Y et al. Gene expression-based, individualized outcome prediction for surgically treated lung cancer patients. Oncogene 2004; 23:5360-70.
[6] Lu Y, Lemon W, Liu P Y et al. A gene expression signature predicts survival of patients with stage I non-small cell lung cancer. PLoS Med 2006; 3:e467
[7] 't Veer L J, Dai H, van de Vijver M J et al. Gene expression profiling predicts clinical outcome of breast cancer. Nature 2002; 415:530-6
[8] Imperatori A, Harrison R N, Leitch D N et al. Lung cancer in Teesside (UK) and Varese (Italy): a comparison of management and survival. Thorax 2006; 61:232-9
[9] Spira A, Beane J E, Shah V et al. Airway epithelial gene expression in the diagnostic evaluation of smokers with suspect lung cancer. Nat Med 2007; 13:361-6
[10] Chomczynski P & Sacchi N. Single-step method of RNA isolation by acid guanidinium thiocyanate-phenol-chloroform extraction. Anal. Biochem. 162:156-9, 1987.
[11] U.S. Pat. No. 5,346,994.
[12] Spira et al. (2004) PNAS USA 101:10143-8.
[13] U.S. Pat. No. 6,128,122.
[14] Potti et al. (2006) N Engl J Med 355:570-80.
[15] Statistical Analysis of Gene Expression Microarray Data. (ed. Speed, 2003). ISBN 1584883278.
[16] Analyzing Microarray Gene Expression Data. (McLachlan et al., 2004). ISBN 0471226165.
[17] Advanced Analysis of Gene Expression Microarray Data. (Zhang, 2006). ISBN 9812566457.
[18] DNA Microarrays and Gene Expression: From Experiments to Data Analysis and Modeling. (Baldi et al, 2002). ISBN 0521800226.
[19] DNA Microarrays, Part B: Databases and Statistics. Volume 411 of Methods in Enzymology.
[20] Microarray Gene Expression Data Analysis: A Beginner's Guide. (eds. Causton et al., 2003). ISBN 1405106824.
[21] Skrzypski (2008)Lung Cancer 59:147-54.
[22] Willey et al. (1997) Am J Respir Cell Mol Biol 17:114-24.
[23] Malard et al. (2007) BMC Genomics 8:147.
[24] Willey et al. (1998) Am J Respir Cell Mol Biol 18:6-17.
[25] Chari et al. (2007) BMC Genomics 8:297.
[26] Geiss et al. (2008) Nature Biotechnol 26:317-25.
[27] Huang et al. (2003) Nature Genetics 34:226-230. Erratum: Nature Genetics 34:465.
[28] British Thoracic Society guidelines on diagnostic flexible bronchoscopy. Thorax 2001; 56 Suppl 1:i1-21
[29] Ettinger D, Akerley W, Bepler G et al. Clinical practice guidelines in oncologyTM. Nonsmall cell lung cancer. Version 1.2007. National Comprehensive Cancer Network (NCCN) 2007.
[30] Mauer E, Baty F, Kehren J, Chibout S D, Brutsche M H. Past, present and future of gene expression-tailored therapy for lung cancer. Personalized Medicine 2006; 3:165-75.
[31] U.S. Pat. No. 6,204,375.
[32] Stamm S, Riethoven J-J M, Le Texier V, Gopalakrishnan C, Kumanduri V, Tang Y, Barbosa-Morais N L, Thanaraj T A. ASD: a bioinformatics resource on alternative splicing. Nucleic Acids Res 2006 34: D46-D55.
[33] Semba S, Iwaya K, Matsubayashi J et al. Coexpression of actin-related protein 2 and Wiskott-Aldrich syndrome family verproline-homologous protein 2 in adenocarcinoma of the lung. Clin Cancer Res 2006; 12:2449-54
[34] Suer G S, Yoruk Y, Cakir E, Yorulmaz F, Gulen S. Arginase and ornithine, as markers in human non-small cell lung carcinoma. Cancer Biochem Biophys 1999; 17:125-31
[35] Eisen M B, Spellman P T, Brown P O, Botstein D. Cluster analysis and display of genome-wide expression patterns. Proc Natl Acad Sci USA 1998; 95:14863-8
[36] Baty F, Facompre M, Wiegand J, Schwager J, Brutsche M H. Analysis with respect to instrumental variables for the exploration of microarray data structures. BMC Bioinformatics 2006; 7:422
[37] Trevino V, Falciani F. GALGO: an R package for multivariate variable selection using genetic algorithms. Bioinformatics 2006; 22:1154-6
[38] Bair E, Tibshirani R. Semi-supervised methods to predict patient survival from gene expression data. PLoS Biol 2004; 2:E108
[39] Bild A H, Potti A, Nevins JR. Linking oncogenic pathways with therapeutic opportunities. Nat Rev Cancer 2006; 6:735-41
[40] Kononen J, Bubendorf L, Kallioniemi A et al. Tissue microarrays for high-throughput molecular profiling of tumor specimens. Nat Med 1998; 4:844-7
[41] Bremnes et al. (2006) Lung Cancer 51:143-58.
[42] Sandler et al. (2006) New Engl J Med 355:2542-50.

1. A method of prognosis of a lung cancer in a human patient, comprising a step of measuring the expression level/s, in a lung tissue sample from the patient, of one or more of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.

2. A method of analyzing a human lung tissue sample, comprising a step of measuring the expression level/s in the sample of one or more of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.

3. A method of analyzing a sample containing RNA transcripts and/or cDNA prepared from a human lung cell, comprising a step of measuring the level/s of RNA transcripts and/or cDNA for one or more of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.

4. The method of claim 1, including a further step of comparing the measured expression level/s to a control level, wherein: (a) an aggregate increase in expression level/s for gene/s (i) to (x) in the sample indicate/s a decreased survival duration relative to the control; (b) an aggregate decrease in expression level/s, or no change, for gene/s (i) to (x) in the sample indicate/s an increased survival duration relative to the control; (c) an aggregate increase in expression level/s, or no change, for gene/s (xi) to (xiii) in the sample indicate/s an increased survival duration relative to the control; and (d) an aggregate decrease in expression level/s for gene/s (xi) to (xii) in the sample indicate/s a decreased survival duration relative to the control.

5. The method of claim 4, wherein the control includes data obtained from a plurality of lung cancer patients having known survival durations.

6. The method of claim 1, wherein expression level/s is/are measured using a nucleic acid array.

7. (canceled)

8. The method of claim 1, wherein the sample was obtained by bronchoscopy.

9. The method of claim 1, wherein at least 1% of cells in a sample are tumor cells.

10. The method of claim 1, wherein the lung cancer is a non-small cell lung cancer.

11. A device comprising immobilized nucleic acid probes for detecting transcripts from two or more of the following 13 human genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.

12. A metagene comprising at least two of the following 13 genes: (i) ARPC2; (ii) SDF2; (iii) AP3D1; (iv) MRPL44; (v) MYO1E; (vi) ARG2; (vii) SNAP29; (viii) HEBP2; (ix) CSNK1A1; (x) CLIP1; (xi) MUS81; (xii) VEGFB; and/or (xiii) OPTN.
